(19) United States
(12) Patent Application Publication
(10) Pub. No.: US 2007/0295327 A1
(43) Pub. Date: Dec. 27, 2007

(54) Olfactory Stimulation

(75) Inventor: Paul Bottomley, Old Basing (GB)

Correspondence Address: SMITH-HILL AND BEDELL, P.C., 16100 NW CORNELL ROAD, SUITE 220, BEAVERTON, OR, 97006, US

(73) Assignee: CAN-DO CORPORATION LTD., Port Louis (MU)

(21) Appl. No.: 11/575,700

(22) PCT Filed: Sep. 21, 2005
(86) PCT No.: PCT/GB05/03618

§ 371 Date: Jun. 22, 2007

(30) Foreign Application Priority Data
Sep. 21, 2004 (GB) 0420925.0

(51) Int. Cl. A61M 11/00; A61M 21/02

(52) U.S. Cl. 128/200.14

(57) ABSTRACT

Use of stimuli in order to provide association with a training regime or other behavior modification technique is known. With regard to olfactory stimulation it will be appreciated that it is necessary that the olfactory is actively provided in the sense the it is not a continuous stimulation but provided in pulses for regular association. Without such distinct presentation of the olfactory stimulus a user may become desensitized. In order to achieve this the present olfactory stimulator comprises a regulator and specific delivery techniques such as the use of a fan or piston to direct the olfactory towards the user's nose.

The present invention relates to olfactory stimulation and more particularly to such stimulation utilised with respect to behaviour modification as a result of training or therapy.

Various techniques with respect to behaviour modification utilise association between a training regime or therapy process and distinct sensory stimulations which act as reminders to an individual as to the training or therapy provided. United Kingdom patent no. 2370233 and International patent application no. PCT/GB97/01898 both outline such training/therapy with associated stimuli response.

Clearly, any form of sensory perceivable stimulation is acceptable as the procedure by which association is made with the underlying training or therapy. Thus, previously noise, light, tactile and olfactory stimuli have been provided in order to achieve the association. Provision of audio and visual as well as tactile stimuli is relatively easy with existing technology, but olfactory devices are more difficult.

It will be understood that to be utilised with respect to so called self help training regimes and therapy it is necessary that the stimulus is discreet and is not generally intrusive with respect to the user individual's normal day to day activities. Thus, although the user individual may perform the training or therapy privately while relaxed in a sitting room or lying on a bed, it is still necessary that the stimulus is not invasive or embarrassing to that individual with respect to noise pollution, etc. With olfactory devices it is also necessary that the scent provided as a stimulus is presented from a conveniently light device and that the scent readily disperses to allow distinct stimuli experiences as well as avoid lingering smells.

In accordance with the present invention there is provided an olfactory stimulator for sustained behaviour modification, the stimulator comprising a scent reservoir and a housing defining a flow path for directing an entrained scent flow, the flow path including regulation means to regulate the entrained scent flow and controlled to allow specific entrained scent flow only as required for association with a stage of a predetermined behaviour modification regime, the regulation means adaptable to a particular user's requirements for the stage of the behaviour modification regime.

Typically, the scent reservoir comprises a cartridge module. Possibly, the scent reservoir is detachable from the housing. Normally, there is a seal between the reservoir and the housing. Generally the seal is an interference fit to provide the seal. Possibly, the housing is entrant upon the reservoir to break a seal for the entrained scent flow.

Possibly, the scent reservoir and the housing are respectively rotatable relative to each other to close the flow path. Generally, the flow path is defined by aligned apertures in the reservoir and the housing.

Possibly, the scent reservoir contains scent gel to provide scent for the entrained scent flow. Possibly, the scent reservoir includes means to excite the scent gel in order to stimulate scent emission. Possibly, the means to excite the scent gel comprises a heater.

Possibly, the scent reservoir and the housing respectively incorporate an interlock detection switch whereby the entrained centred flow is only allowed when the interlock detection switch is closed.

Possibly, more than one scent reservoir are coupleable to the stimulator in order to provide by specific selection a more distinct entrained scent flow for association with individual user's desires for behaviour modification.

Typically, the flow path is indirect and/or labyrinthine to regulate entrained scent flow. Possibly, the flow path incorporates at least one chamber to contain scented air for immediate flow towards an individual and/or limit inadvertent leakage of scent from the stimulator. Generally, the path incorporates means to project the entrained scent flow. Typically, the means to project the entrained scent flow comprises a piston. Possibly, the piston is driven by an electrical wire expansion or contraction. Alternatively, a solenoid or electrical motor could be used to drive the piston. Possibly, the means to project the entrained scent flow comprises a drawing suction fan.

Typically, the flow path incorporates directional jets for providing directional projection of the entrained scent flow.

Typically, the flow path incorporates a specifically operable flow control valve.

Also in accordance with the present invention there is provided apparatus for behaviour modification including means to train or provide therapy to an individual incorporating provision of control signals associated with sections of that means to provide training or therapy to an individual, the control signals being operable to control an olfactory stimulator as described above whereby the entrained scent flow provides association with the training or therapy.

Possibly, the means to project a scent includes a fan or piston or bellows. Alternatively, the means to project the scent flow includes a piezo-electric crystal vibration or nebulisation process for entraining the scent flow.

Embodiments of the present invention will now be described by way of example only and with reference to the accompanying drawings in which:—

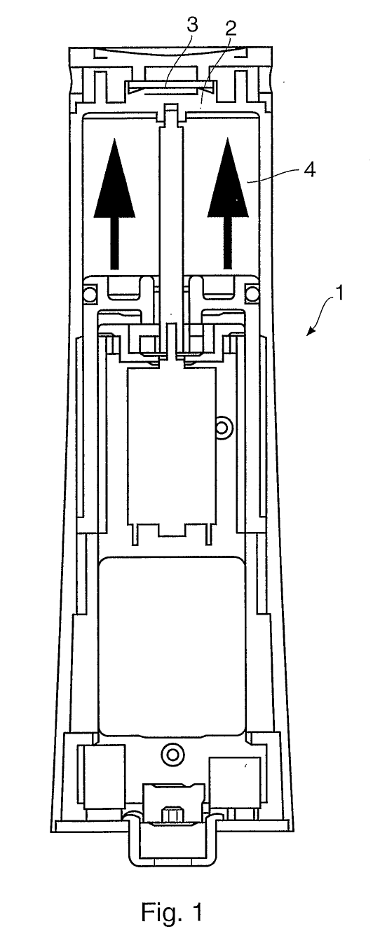

FIG. 1 is a schematic cross-section of an olfactory stimulator in accordance with a first embodiment of the present invention;

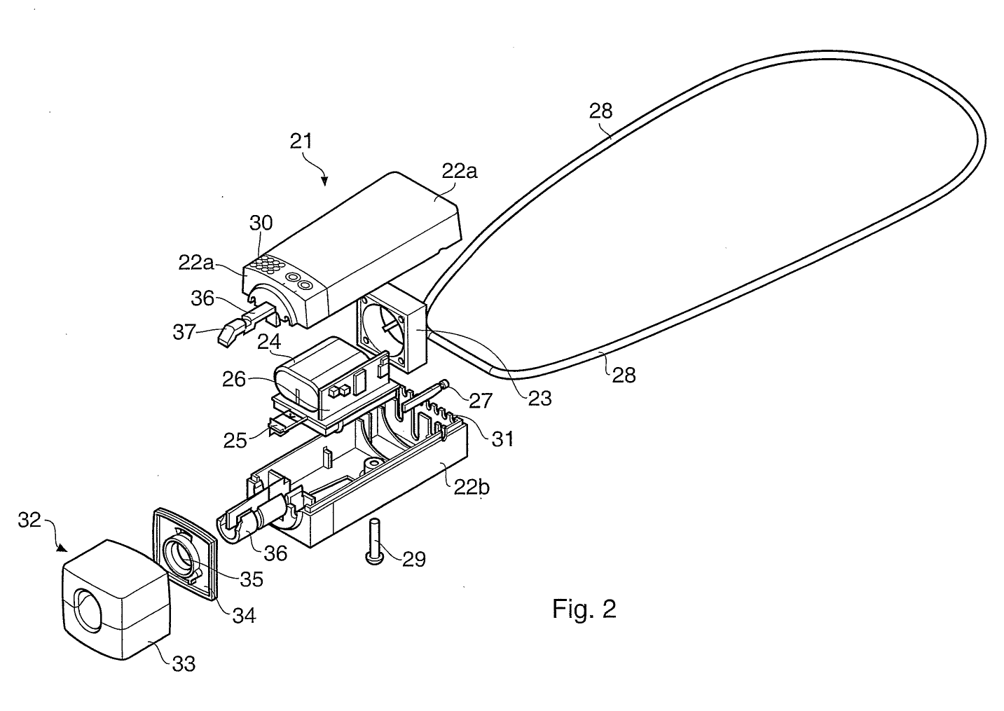

FIG. 2 is an exploded view of an olfactory stimulator in accordance with a second embodiment of the present invention;

FIG. 3 is a longitudinal cross-section of an assembled stimulator in accordance with the second embodiment depicted in FIG. 2 illustrating scent flow;

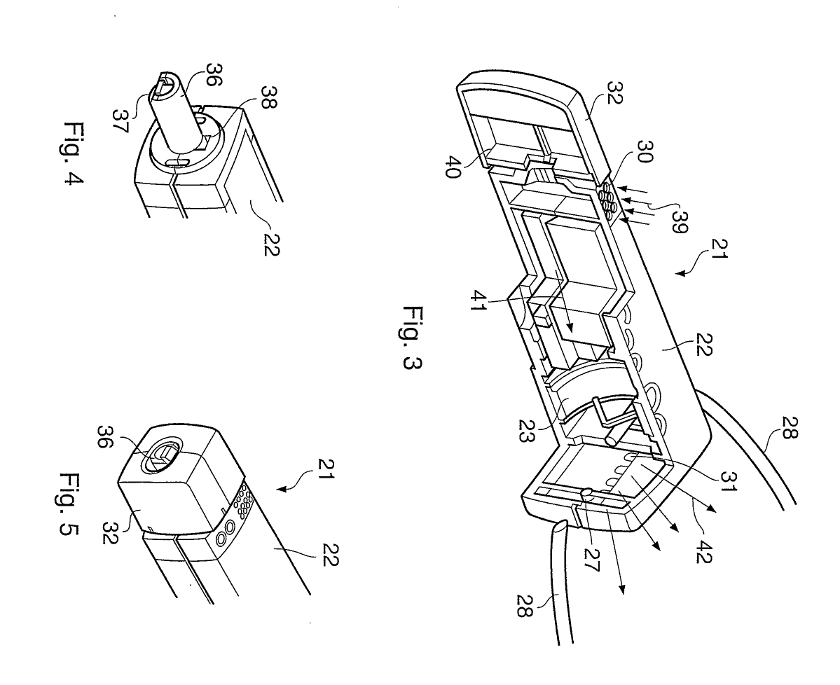

FIG. 4 illustrates a housing end for a stimulator in accordance with the second embodiment of the present invention depicted in FIGS. 2 and 3;

FIG. 5 is an illustration of a scent reservoir module secured to the housing depicted in FIG. 4;

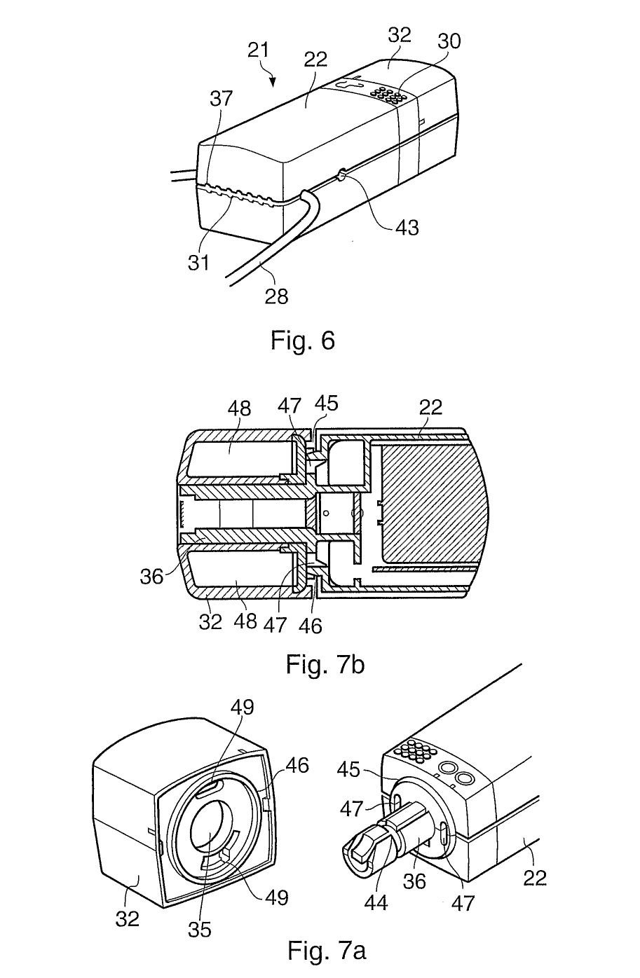

FIG. 6 illustrates an assembled stimulator;

FIG. 7 illustrates an interference seal between a stimulator housing and scent reservoir module;

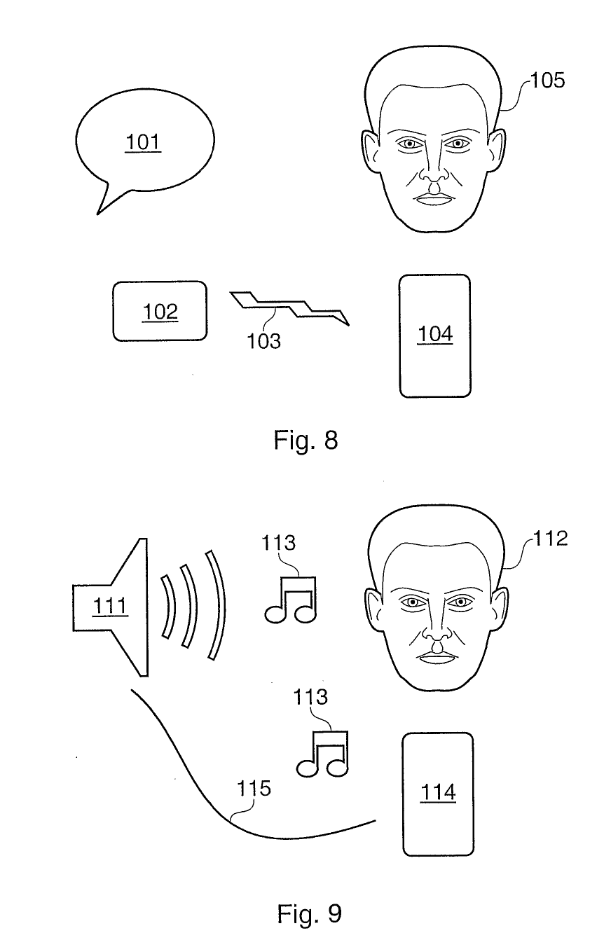

FIG. 8 illustrates a first behaviour modification system in accordance with the present invention;

FIG. 9 illustrates a second behaviour modification system in accordance with the present invention;

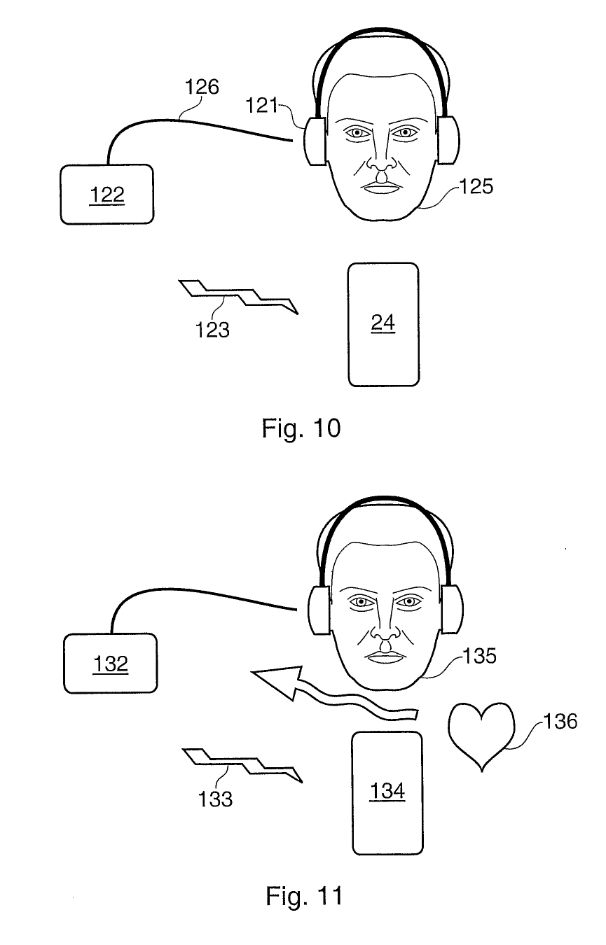

FIG. 10 illustrates a third behaviour modification system in accordance with the present invention; and,

FIG. 11 illustrates a fourth behaviour modification system in accordance with the present invention.

Techniques utilising association between a stimuli and a training regime or therapy are known as described above. In order to utilise an olfactory scent as the stimulus, it is necessary to provide a suitably small yet robust device which can be carried by the user whilst also providing sufficient scent to be perceivable by that user. Clearly, it is also necessary that the stimulator device allows projection of a scented flow towards a user after receiving an instruction signal. To avoid conflict with neighbours and other non users it should also be understood that the device should be subtle and non intrusive. In these circumstances it is necessary to entrain a scent flow such that that flow is directed towards the nose of a user or at least towards that user's face in a discreet amount such that the scent provides the desired reminiscence of the training or therapy for the user. Generally, the scent can be projected in a number of ways provided that there is dosing of the scent within the entrained flow. Thus, the entrained scent flow may be generated by a fan or a piston or a bellows or a combination of these. Alternatively, piezo-electric crystal vibration or nebulisation may be utilised in order to entrain the flow projected towards a user's face.

FIG. 1 illustrates a piston type system in which high pressure is created in order to propel the scent flow towards the face of a user. Thus, the stimulator 1 operates by providing a chamber 2 with a diaphragm seal 3 at its top end. When a piston 4 operates the seal 3 is pushed open under pressure giving a fine high velocity jet of scent entrained air towards a user. When the piston 4 returns the seal 3 closes. Such an arrangement reduces scent leakage into the atmosphere when the stimulator 1 is not being used. It will be appreciated that continued scent leakage would desensitise a user to the associated effect of the olfactory stimulus reminiscent of the training or therapy regime. As the stimulator 1 will generally be utilised close to a user's nose, it will be understood that even relatively small continued leakage of the scent will be appreciated by the user and therefore may present a particular desensitising effect upon that user.

As an alternative to utilising the piston in order to drive the entrained scented flow towards the user it will be appreciated that a lower pressure system may be used. In such an arrangement the seal itself would be mechanically opened. Operation of the mechanical seal may be through direct user action or by receipt of a control signal associated with the training or therapy regime as a recorded signal or upon a trainer or therapist's particular action through a remote control fob device and subsequently by the user. It will be understood that the piston movement or seal opening could be achieved through use of a so called muscle wire comprising a lightweight electrical wire which expands and contracts through electrical heating. Alternatively, a solenoid or electrical motor through a spindle and drive piston movement as required could be used. Nevertheless, whichever technique is used, it is important that there is no leakage of the scent in order to avoid any potential for desensitising of the user.

As indicated, a relatively small quantity of scent is provided in the entrained scented flow towards the user. In such circumstances it is important that the entrained scented flow is at least substantially directional to maximise its effect. This directionality is typically achieved through use of jets and nozzles which appropriately project the entrained scented flow towards the nose of a user. However, further features such as stand off sets can be utilised to hold the stimulator off the chest of a user or for direction when used from a surface such as a table top.

Preferably, a stimulator is located close to a user's nose and so will typically be hung around the neck or worn as a necklace about the user. Clearly, the closer the stimulator is to the nose the less scent is required which in turn will lead to less environmental dispersion and also faster appreciation by the user and subsequent dispersal. This will leave greater distinctiveness to the scent stimulation for repeated perceptible association by the user with the underlying training or therapy. Unfortunately, such close association of the stimulator to the user's nose also renders any inadvertent leakage of the scent when not required as being more likely to be perceived by that user. In such circumstances where appropriate and also in order to avoid any intrusive weight or size problems with respect to the stimulator it may be advantageous to locate the stimulator away from the user's nose mounted upon a waist belt with a connecting conduit tube from that stimulator to take the entrained scent flow for release adjacent to the user's nose through a mask. In such circumstances it is unlikely that any incidental scent leakage would leak upwards in the tube towards the user and any stimulator weight or size problems would be avoided.

Additionally, it may be advantageous to combine the olfactory entrained scent flow stimulus to a user for training or therapy association with a more conventional audio or light visual indication or vibration for stimulation via another device. In such circumstances the stimulator may incorporate a buzzer or LED light flash or vibration to act in concert with the released olfactory entrained scent flow. This approach may also provide a conscious or subconscious indication to the user that it is time to perform a deeper inhale in order to receive the necessary olfactory scent stimulation.

Displacement type projection of an entrained scent flow may be relatively simple but generally may create difficulties with respect to short term sustain of the scent flow towards the user. In such circumstances as described in the second embodiment depicted in FIGS. 2 to 7, it may be more appropriate and dimensionally efficient to provide for a fan projection of the entrained scented flow towards the user. Thus, an olfactory stimulator will generally comprise a housing 22 in a two part form 22a, 22b arranged to encapsulate a fan 23 and means to drive that fan 23 including a battery 24, circuitry 25 to receive control signals in order to drive the fan 23 and appropriate radio frequency circuitry 26 in order to receive those control signals. Typically, an LED light 27 is also provided in order to indicate operational status for the stimulator 21 to a user. The stimulator 21 is held about a user's neck using a lanyard cord 28 which generally passes through the housing 22 which is held together using a screw 29. The housing defines a flow path through which air is sucked through inlet apertures 30 and out of jets 31 towards a user.

The housing 22 is associated with a scent reservoir cartridge or nozzle 32 comprising a casing 33 and a lid 34. The reservoir 32 generally contains scent in a gel form such that the airflow stimulated by the sucking action of the fan 23 draws scent into the entrained flow passing through the inlet 32 and out of the jets 31 towards the user. Alternatively, the scent may be provided by an impregnated card or scented crystals or a liquid reservoir appropriately presented. Possibly, the scent may be heated to create scent vapour to facilitate scent entrainment within the air flow. This heating may be through an electrical heater (not shown) within a scent reservoir 32.

As indicated above, the principal basis upon which a stimulator operates is in order to release an olfactory scent upon demand to a user. Clearly, it is necessary for the scent to be characteristic and transient as well as distinguishable or unique for the user in order to appreciate the stimulation. In such circumstances, it is desirable as illustrated with respect to the second embodiment that the scent reservoir 32 is provided which can incorporate a range of differing scent types as required by the user. The stimulator incorporating the fan 23 and appropriate controller circuitry in order to ensure release of olfactory scent as required will generally be designed to allow the stimulator to be hung around the neck of a user. Upon demand, the fan 23 is activated and air is sucked over the scented gel within the scented reservoir 32. An entrained scented flow is then created which is projected through the jets 31 towards the user. Typically, for convenience, as indicated above, a remote control through a radio frequency control loop will be utilised such that the fan 23 only becomes operational when required. Alternatively, a simple switch may be associated with the stimulator to allow manual operation of the stimulator device. Similarly, simple timer circuitry or response to signals built into a music or computer or phone message may also be utilised in order to operate and control fan action in order to generate the desired entrained scented flow towards the user.

The use of a scent reservoir in a cartridge or modular form allows a self sealing arrangement to be utilised such that the possibility of scent leakage or flood is minimised avoiding any desensitisation by the user to that particular olfactory scent. Normally, the scent reservoir as indicated will take the form of a cartridge or module delivered with a seal over an outlet 35 which will be removed just prior to use. To attach the scent reservoir, it is orientated into an OFF position and simply pushed into place through assembly upon a shaft 36 of the housing 22. The shaft 36 enters the scent outlet 35 such that a retaining clip 37 engages the scent reservoir 32 to retain association between that reservoir 32 and the housing 22. As will be described later, typically an interference fit is created in order to act as a seal barrier between the housing 22 and the scent reservoir 32.

The scent reservoir 32 is located on the shaft 36 and prevented from moving by the clip 37.

FIGS. 4 and 5 illustrate respectively an end of the housing 22 and assembly of that housing 22 with the scent reservoir 32. As indicated, an interference fit is provided in order to act as a seal, but the scent cartridge is allowed to rotate upon the shaft 36. In such circumstances a detector switch or interlock 38 acts to provide an indication as to scent reservoir attachment as well as position. The switch 38 generally operates by displacement upon a cam face of the scent reservoir cartridge.

Assembly of the scent reservoir cartridge upon the shaft 36 is only allowed in an OFF position. That is to say where there is no path from the scent reservoir to the flow path through the housing 22 and onward projection to the user. In such circumstances the leakage of the scent is avoided.

In order to operate the stimulator generally the scent reservoir cartridge 32 is rotated such that the switch 38 indicates an ON position to the control device within the stimulator 21. In the ON position the scent reservoir cartridge 32 and in particular its lid 35 formed with an intentional interference with a flange on the base of the housing 32 creating a simple seal. However, in this position, respective holes in the scent reservoir cartridge and in the base of the housing 32 are aligned in order to create the flow path from the scent reservoir through the housing 22. Essentially, a passage is opened for air to pass from the outside of the stimulator 21 via inlets 30 through an air space above the scent gel within the reservoir 32 and then through the housing 22 and out of the jets 31 towards a user. This flow passage or pathway is intentionally designed to be indirect and labyrinthine and will normally incorporate two relatively large sump chambers. These sump chambers act as further leakage draught proofing in order to reduce the likelihood of scent escaping from the product place accidental airflow rather than as specifically stimulated by the sucking action of the fan 23.

FIG. 3 provides as a simple longitudinal cross-section an illustration of the flow path. Thus, air is drawn through the inlets 30 in the direction of arrowheads 39 by a fan 23 such that scent is drawn from the reservoir 32 along with the air flow 39 in the direction of arrowhead 40 such that a combined flow and an entrained scented flow 41 then passes through the housing 22 and out of the jets 31 in the direction of arrowheads 42 towards a user.

Generally, the LED 27 will pulse to indicate when the stimulator 21 is operational. When operational the fan 23 will rotate in order to create the sucking positive air pressure through the pathway whereby there is an entrained scented air flow out of the jets 31 towards the user. As indicated previously by using a fan rather than a piston, it will be understood that the fan can operate for a pre set time dependent upon the control signals provided such that an appropriate scent dosage is presented to the user whilst avoiding excessive scent release which can create desensitising effects upon the user and also create scent pollution. Clearly, the LED by a pulsing light effect acts as a secondary indicator to the user which may consciously or sub-consciously generate a deeper inhale by that user further enhancing the olfactory stimulating effect.

Once the user is finished, the system is simply switched off by reverse rotation of the scent reservoir cartridge 32 about the shaft 36 such that the holes in that cartridge and a base of the housing 22 are no longer aligned. In such circumstances scent leakage is minimised. It will also be understood that when the scent reservoir is removed from the housing 22 the stimulator is essentially turned off and a physical seal barrier can then be again placed across the outlet 35.

In order to extend operational life it will be understood that if the stimulator is left operational for a period of time without receiving a control signal then it will enter a standby mode. In this mode in order to preserve battery life the stimulator will no longer operate unless firstly switched to an OFF position again for subsequent entry into an operational state by rotation of the scent reservoir 32 about the shaft 36. This procedure is detected by the switch 38.

FIG. 6 illustrates an assembled stimulator 21 in accordance with the second embodiment of the present invention. Thus, the stimulator 21 is presented upon a lanyard cord 28 with the housing 32 secured upon a reservoir cartridge 32 such that air is drawn through inlets 30 and passes out of jets 31 towards a user. An LED 37 provides a pulsed light signal indicative of olfactory stimulator 21 operation, which as indicated previously will act as a reinforcement to the user as to the desired stimulation and therefore association with the training or therapy regime. Within the stimulator 21 as indicated there is a fan driven by a battery. To avoid the necessity of using disposable batteries a charge socket 43 is provided to allow a rechargeable battery within the stimulator 21 to be used.

Of great importance is the avoidance of scent leakage which as indicated will create problems with respect to desensitisation of a user individual as well as potential conflicts with neighbours, etc. Thus, as illustrated in FIG. 7 an intentional interference fit is provided between the scent reservoir cartridge 32 and a housing 22 whereby the shaft 36 acts through a location slot 44 to enter a scent outlet 35 to achieve positioning of the housing 22 relative to the scent reservoir cartridge 32. An interference seal is created by a flange 45 at the base end of the housing 22 engaging about a rim 46 in the cartridge 32. An angled face between the flange 45 and rim 46 acts to give a tolerance range to the assembly, but nevertheless it is important that location between the housing 22 and the scent reservoir cartridge 32 is sufficiently accurate that the best possible seal is achieved whilst allowing the cartridge 32 to rotate between the ON and OFF positions as described previously. Additionally, or alternatively, an O ring seal may be used to provide a seal.

FIG. 7b illustrates the assembly of the scent reservoir cartridge 32 upon the shaft 36 in an OFF or closed position such that holes 47 in the base of the housing 22 are not aligned with reciprocal holes in the scent reservoir cartridge 32. In such circumstances gel in chambers 48 cannot enter the housing 22 and therefore pass along the flow path for entrained centred flow through that housing 22 for projection towards an individual as described previously. It will be necessary for holes or apertures 49 (FIG. 7a) to become aligned with the holes 47 in the housing 22 in order to create that continuous path for the entrained centred flow as described previously stimulated by fan 23 operation. It is rotation about the shaft 36 which creates alignment between the apertures 49 and the holes 47 so that the seal created by the interference association between the flange 45 and rim 46 must still allow such rotation for operation of the stimulator 21 in accordance with the present invention.

As indicated above, the present olfactory stimulator is useful with respect to creating associations between the olfactory scent and a training or therapeutic regime. In such circumstances it is important that there is a signal association between the stimulator and that training regime. In such circumstances the signal can be created manually but more normally will become part of a recorded message or process. The stimulator must therefore be locked to the particular source of signal. In individual sessions, exclusivity of the link between the training or therapeutic regime and the stimulator may not create too great a problem but where there are a group of users or a number of users requiring different stimuli at different stages, problems may be created. In such circumstances a fixed link between a control fob which either manually or through monitoring/interrogation of the recorded training or therapeutic regime acts to pass the control signal to the stimulator in order to release the olfactory scent as described may be provided. Alternatively, the association between the controller in the form of a fob or other monitor/interrogator and the stimulator may be locked for simply one session, that is to say until the stimulatory switch is switched off. These arrangements allow a situation where the user and/trainer or instructor can be given exclusive rights to operate the stimulator as required for the training or therapeutic regime.

In order to further enhance the distinctiveness and uniqueness of the olfactory scent, as indicated above, each scent reservoir in the form of a cartridge may contain a unique blend of scents for that particular user. The user may pick their own particular scent from the range available in cartridges or alternatively two or more cartridges could be secured upon a single housing such that each cartridge provides its own element to the combined olfactory scent entrained through the flow path for projection to that user for appropriate stimulation. It should be understood that scent mixes will be designed specifically to give a clear and distinct smell and not a confused message. Furthermore, the scent utilised must be transient to reduce the risk of the user becoming desensitised to the olfactory scent or as a result of creating a general scent pollution for third parties about the user. These features are achieved through utilisation of a good sealing regime and as indicated above only provision of a limited amount of scented air for stimulation to a user. Finally, the scent may be formulated to show rapid decay when dispersed in air thus reducing the chance for polluting neighbours to a user.

A high pressure system has advantages with respect to obtaining a clear and directed entrained scented flow which is less likely to be dispersed before being smelt by a user and able to travel greater distances, but may create problems with respect to environmental loss of scent. In such circumstances such high pressure systems may be particularly advantageous with respect to utilisation of conductor tubes from a belt mounted stimulator in accordance with the present invention to the vicinity of a user's nose. It will also be understood that using high pressure systems based upon a displacement piston against a seal diaphragm ensures that any seal will simplify sealing arrangements by only ensuring the seal is opened under pressure. With lower pressure systems utilising a fan, typically a mechanical or manual system of opening a seal as described above through rotation of a seal reservoir cartridge will be necessary to reduce leakage when not operational. Similarly, by provision of sump chambers within the flow path, it will be appreciated that there is a resistance to leakage of scent from the stimulator when not specifically forced by the action of a piston or a fan. These sump chambers will also act as priming chambers to allow the scent to diffuse and dose the air between operations. That is to say, an entrained scented flow will be quickly presented to the user immediately rather than there being an initiation phase before appropriate entrained scent flow concentration is achieved. Essentially the scent will concentrate in the sump chambers during times of non operation and this concentration will then be immediately drawn by the fan or piston for presentation to a user. However, rapid repeat usage may lead to a perceived reduction in scent strength with each usage but this is balanced by the benefit of the necessity of only a relatively small amount of scent for a highly charged small volume of scent air upon first activation.

Nozzles or jets will be utilised in order to ensure appropriate directional presentation of the entrained scented flow to the user, that is to say about their face and particularly their nose.

It will be appreciated that the present olfactory stimulator could also be utilised with other forms of stimulation as indicated previously such as audible sounds or phrases along with light crystals or physical tactile vibration, etc. Thus, for example, a head set may be provided in which earphones connected through wiring to a sound reproducing device such as a CD player may be combined through operationally rigid but otherwise malleably adjustable presenter at the end of which an olfactory stimulator in accordance with the present invention is secured. Thus, the user can receive audio stimulation through the earphones and olfactory stimulation through the present olfactory stimulator. Operation of the olfactory stimulator may be through a separate radio frequency coupling whereby control signals are sent either by a manual fob or through monitoring of a training regime self help tape or CD. Alternatively, hard wiring which will normally be coupled to the earphones may also incorporate control signals for the olfactory stimulator. In such circumstances the head set would resemble a normal head set such as those used by pilots or telephony operators. Furthermore, the present olfactory stimulator could be made of a size such that it can piggy-back upon an existing microphone of a head set. Such a head set would therefore be less intrusive and allow behaviour modification utilising a number of procedures including single stimulus, i.e. audio or olfactory, etc or discreet stimulus phases combining two or more stimulations in appropriate time spaced or overlapping association to create an individual uniqueness with respect to the stimulation provided for association with the underlying training or therapy regimes.

The stimulator may “learn” its control trigger for each individual fob in a similar manner to a motor vehicle central locking fob. Alternatively, the stimulator may be controlled by a number of fobs selectively. Thus, if you are at a therapist your stimulator can recognise the therapist's as well as your device, but only respond to the therapists' device during a training session. Of particular note are self help systems for multiple users. Two people can be in a house, one operating their stimulator manually, the other listening to a self help system and thus the stimulator operates automatically from signals say from the CD of the self help system. Neither system interferes with the other as they are locked onto two different control signals. If the manual user now wants to join in then they simply switch their stimulator off then on and when it hears the learn signal from the self help system it will lock onto it for that session. Thus, the stimulator is simply locked to a particular control fob for one session.

Activation of the olfactory stimulator can be achieved in a number of ways. Examples of these approaches are outlined below.
a) It is possible the olfactory stimulator is activated simply by a fob device which emits a radio frequency signal when the fob button is depressed. This radio frequency signal, which may be digital or otherwise, is as unique as possible for a particular stimulator, and will be picked up in order to activate that olfactory device for an olfactory release regime.
b) The stimulator releases olfactory stimulation automatically when an appropriate control signal, possibly in the form of sound or Blue Tooth control or infra red or any other means, is either wireless or hardwire, identified in a recorded message played by a separate player as required.
c) A fob is provided in which there is appropriate means for playing the recorded message which in itself then issues the necessary control signals to activate the olfactory stimulator for use of the olfactory stimuli; or,
d) By appropriate two way control connection between a fob and the stimulator it will be understood that status information with respect to the user can be transmitted back to the control fob and message playing modes, in terms of speed, repetition etc, and can then be adjusted as necessary in order to achieve the desired signal to activate the stimulator adjusted for best effect.

Control in terms of activation and adjustment of olfactory release can be achieved by providing apparatus couplings in any appropriate way. Thus, radio, TV, mobile phone, terrestrial phone, computers and any means of playing a message whether it be on a cassette tape, CD, MP3 or other means can then be provided in order to activate the appropriate signals to control release of the olfactory stimuli from the stimulator.

Couplings between systems for behaviour modification incorporating an olfactory stimulator or other device can be hard wired or utilised in any wireless coupling including for example as illustrated above, radio frequency connection or blue tooth standard handshake control signals or infra red or sound or pressure or other wireless control regime.

It will be appreciated that normally the control signals between the olfactory stimulator and the control fob will generally take the form of radio frequency pulses therebetween. However, it is also possible to operate the stimulator remotely via a mobile phone coupling. In such circumstances the stimulator can have its own mobile phone receiver including a phone SIM card and receive instructions directly over an existing phone network through dial up, etc. Furthermore, upgrading of the stimulation control regime could be achieved through downloading appropriate re-programming details via that mobile phone linkage.

Additionally, provision may be made within the behaviour modification system and in particular the control regime whereby there is an intelligent degree of feedback to the user's actual physical condition/symptoms at the incident time. Thus, for example a heart or breathing monitor or thermometer could monitor the user and only allow operation of the stimulators for a session of behaviour modification when the subject user is sufficiently relaxed.

In addition to user feedback it will also be appreciated that operational data could also be exchanged or represented in order to improve operation of the system. Thus, an indication as to low battery charging may be provided in the form of warning light or a pre-recorded audio warning or simply curtailing the time period of a training or therapeutic session. Furthermore, if the system is being over used and therefore the user risks desensitisation, similarly a warning message or indicator may be provided. It will also be understood with respect to monitoring that a therapist or trainer may be provided with an indication as to the number of users logged into the system, who is actually receiving stimulus at any one time and possibly a confirmation that the olfactory stimulus is actually operating. In such circumstances both the users and the trainer/therapist will be able to maximise or tailor the training regimes as required.

It will be appreciated that the stimuli utilised with respect to the present invention can be delivered in a number of systems. The drawings illustrate four exemplary systems but others may be used.

FIG. 8 illustrates a first manual, behaviour modification system in which a stimulation device 104 is activated by a fob 102 which emits an RF signal 103 when a fob 102 button is pressed. The RF signal 103 is picked up by the device 104.

A therapist or trainer 101 speaks directly to a user 105. When it is time to activate the secondary stimulation, in this case the device 104 the fob 102 is manually activated and a signal 103 is transmitted. Alternatively, the system may be a self-help regime where a CD message is played with prompts to manually activate the fob 102.

The signal 103 can take many forms including radio waves, infra red or sound. Of note is a similar scenario where the trainer is communicating by a phone or similar and the signal is transmitted also via a phone network.

FIG. 9 illustrates a second, audio, behaviour modification system in accordance with a standard player device such as a CD player 111, broadcasts a lesson. Within this lesson there is a trigger signal 113 which could be in the form of a jingle, which is heard by a user 112. Similarly this trigger signal 113 can be ‘heard’ by the stimulator device 114, or by a device such as a fob (see FIG. 1). This will cause the device 114 to activate (in the case of a fob via RF). Alternatively a wire 115 could run from the output socket on the CD player 111. This would reduce the effects of environmental pollution, passing the signal to headphones on the user and/or directly to the device 114 or a trigger device (fob).

FIG. 10 illustrates a third integral behaviour modification system in accordance with the present invention. A player device 122 contains the means for playing a recorded message.

A user 125 could wear headphones 121. The player device 122 replaces a CD player and the fob from the systems above. The device 122 could be an MP3 player or any similar device that can store and broadcast sound/vision information to the user 125. However at the appropriate parts in the broadcast message the device 122 transmits a signal 123 to a stimulator device 124. Again this signal 123 can take many forms, including a radio frequency. The device 122 is also likely to have a manual operation button, enabling it to be used like a simple fob (see FIG. 1).

It is also of note the player device 122 could be a home PC or any electrical device capable of being programmed and sending a signal. This includes modern mobile phones. Finally, the headphones 121 could be coupled to the player device 122 by a fixed wire 126 or any wireless link.

FIG. 11 illustrates a fourth, feedback behaviour modification system in accordance with the present invention. A connection, whether wire or wireless between a fob 132 and the stimulator device 134 is two-way. Thus, bodily information from a user 135 can be transmitted back to the fob 132 and the message playing modes (speed/repetition etc) can be adjusted as necessary and a signal 133 to activate the stimulus device 134 adjusted accordingly.

A secondary device 136, such as a heart beat or body temperature sensor monitors environmental or user attributes and feeds these back to the fob 132 as a control device. In this example the users heart rate is broadcast and the stimulus phase adjusted accordingly.

As indicated above, the present invention is based upon creating environmentally distinct stimulation phases. Thus, these phases may combine an audio or visual stimulation created by a radio, TV, mobile phone, terrestrial phone, computer or player for cassette tapes, CD, MP3 or other programmable recall apparatus along with stimuli created by vibrators or olfactory stimulators, lights, crystals or temperature or otherwise. Communication between all parts of the equipment to create the distinct stimuli, in sequence, and so create the stimulus phase may be hard wired together or by use of wireless technology, such as radio frequency communication, or Blue Tooth, or infra red, or ultrasonic, or other means of coordinating stimuli emissions to a user.

Finally, it will be appreciated that the displacement length and therefore entrained scented flow volume or rate of fan rotation may be utilised in order to adjust the absolute volumes or rate of entrained scented flow release for different circumstances dependent upon user requirements. Additionally, nose pieces or masks may be utilised as described above in order to further enhance good presentation of the entrained centred flow to the user about their nose whilst avoiding leakage.

It will be understood that actuation signals for the apparatus may be embedded in to a recording or message on a CD or other recording medium. Thus, the stimulator will be activated when those actuator signals are detected in an auto actuation regime.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1-29. (canceled)

30. An olfactory stimulator for sustained behavior modification, the stimulator comprising a scent reservoir and a housing defining a flow path for directing an entrained scent flow, the flow path including regulation means to regulate the entrained scent flow and controlled to allow specific entrained scent flow for pre-determined time periods for association with a behavior modification step, the scent reservoir within the stimulator arranged to be substantially configured only to allow scent release to the flow path to inhibit environmental scent leakage.

31. A stimulator as claimed in claim 30 wherein the scent reservoir comprises a cartridge module.

32. A stimulator as claimed in claim 30 wherein the scent reservoir is detachable from the housing.

33. A stimulator as claimed in claim 30 wherein there is a seal between the reservoir and the housing.

34. A stimulator as claimed in claim 33 wherein the seal is an interference fit to provide the seal.

35. A stimulator as claimed in claim 30 wherein the housing is entrant upon the reservoir to break a seal for the entrained scent flow.

36. A stimulator as claimed in claim 30 wherein the scent reservoir and the housing are respectively rotatable relative to each other to close the flow path.

37. A stimulator as claimed in claim 30 wherein the flow path is defined by aligned apertures in the reservoir and the housing.

38. A stimulator as claimed in claim 30 wherein the scent reservoir contains scent gel to provide scent for the entrained scent flow.

39. A stimulator as claimed in claim 38 wherein the scent reservoir includes means to excite the scent gel in order to stimulate scent emission.

40. A stimulator as claimed in claim 39 wherein the means to excite the scent gel comprises a heater.

41. A stimulator as claimed in claim 30 wherein the scent reservoir and the housing respectively incorporate an interlock detection switch whereby the entrained scent flow is only allowed when the interlock detection switch is closed.

42. A stimulator as claimed in claim 30 wherein more than one scent reservoir are coupleable to the stimulator in order to provide by specific selection a more distinct entrained scent flow for association with individual user's desires for behavior modification.

43. A stimulator as claimed in claim 30 wherein the flow path is indirect and/or labyrinthine to regulate entrained scent flow.

44. A stimulator as claimed in claim 30 wherein the flow path incorporates at least one chamber to contain scented air for immediate flow towards an individual and/or limit inadvertent leakage of scent from the stimulator.

45. A stimulator as claimed in claim 30 wherein the path incorporates means to project the entrained scent flow.

46. A stimulator as claimed in claim 45 wherein the means to project the entrained scent flow comprises a piston.

47. A stimulator as claimed in claim 46 wherein the piston is driven by an electrical wire expansion or contraction.

48. A stimulator as claimed in claim 47 wherein a solenoid or electrical motor could be used to drive the piston.

49. A stimulator as claimed in claim 45 wherein the means to project the entrained scent flow comprises a drawing suction fan.

50. A stimulator as claimed in claim 30 wherein the flow path incorporates directional jets for providing directional projection of the entrained scent flow.

51. A stimulator as claimed in claim 30 wherein the flow path incorporates a specifically operable flow control valve.

52. Apparatus for behavior modification including means to train or provide therapy to an individual incorporating provision of control signals associated with sections of that means to provide training or therapy to an individual, the control signals being operable to control an olfactory stimulator as claimed in claim 30 whereby the entrained scent flow provides association with the training or therapy.

53. Apparatus as claimed in claim 52 wherein the means to project a scent includes a fan or piston or bellows.

54. Apparatus as claimed in claim 52 wherein the means to project the scent flow includes a piezo-electric crystal vibration or nebulization process for entraining the scent flow.

55. Apparatus as claimed in claim 52 wherein the apparatus includes means to detect an actuation signal for the apparatus in a recorded message to allow auto actuation of the apparatus and in particular the stimulator.
